(19) United States
(12) Patent Application Publication
(10) Pub. No.: US 2007/0123904 A1
(43) Pub. Date: May 31, 2007

(54) Distraction instrument and method for distracting an intervertebral site

(75) Inventors: Shawn D. Stad, Fall River, MA (US); Patrick Fatyol, Whitman, MA (US); Mark Gracia, Rochester, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/263,583

(22) Filed: Oct. 31, 2005

(51) Int. Cl. A61F 2/00

(52) U.S. Cl. 606/99

(57) ABSTRACT

A distraction instrument includes a drive rod, a driver mechanism coupled to the drive rod, and a pair of arms linked to the driver mechanism. A method of implanting an artificial disc or a fusion implant in an intervertebral site includes preparing the intervertebral site, actuating a gear mechanism of a distraction instrument to distract the intervertebral site, and inserting at least a core of the artificial disc or fusion implant into the intervertebral site.

### BACKGROUND OF THE INVENTION

Spinal surgery involves many challenges as the long-term health and mobility of the patient often depends on the surgeon's technique and precision. One type of spinal surgery involves the removal of the natural disc tissue that is located between adjacent vertebral bodies. Procedures are known in which the natural, damaged disc tissue is replaced with an interbody cage or fusion device, or with a disc prosthesis.

The insertion of an article, such as an artificial disc prosthesis, presents the surgeon with several challenges. The adjacent vertebral bodies collapse upon each other once the natural disc tissue is removed. These bodies must be separated to an extent sufficient to enable the placement of the prosthesis. However, if the vertebral bodies are separated, or distracted, to beyond a certain degree, further injury can occur. The disc prosthesis must also be properly positioned between the adjacent vertebral bodies. Malpositioning of the prosthesis can lead to pain, postural problems and/or limited mobility or freedom of movement.

Instrumentation that uses linked mechanisms to distract the disc space has been developed. For example, one end of a distracting spreader is threaded and screwed into an internal threaded opening in the spine. Such threaded instruments can provide the surgeon with a mechanical advantage for slowly distracting the disc space by pushing a spreader through a hollow tube. Using this convention, however, leads to a slow distraction that requires the surgeon to turn the instrument many times to obtain the desired distraction. Further, the repeated turning motion can cause the instrument and implant to shift out of alignment during the distraction procedure.

Similarly, as disclosed in U.S. Pat. No. 6,755,841, a surgeon may apply an alternative method of distraction by impacting a spreader between two blades or rails of a distraction instrument. Impaction, however, is a less desirable means of distraction because it is less controlled and distraction occurs at a sudden and an increased rate. The result can be undesired overdistraction or damage to the neural tissue. Furthermore, some parallel distraction-type instruments force the surgeon to impact the implant endplates into the disc space without a distraction aid, and distraction of the disc space is only provided for the implantation of the implant core.

Greater accuracy and precision are critical for an artificial disc. Artificial discs offer several theoretical benefits over spinal fusion for chronic back pain, including pain reduction and a potential to avoid premature degeneration at adjacent levels of the spine by maintaining normal spinal motion. However, like spinal fusion surgery, surgical techniques and procedures do not always work reliably for artificial disc implantation. Despite existing tools and technologies, there remains a need to provide a device to facilitate the proper and convenient insertion of an object, such as a disc prosthesis, between adjacent vertebral bodies while minimizing the risk of further injury to the patient. There remains a need for improved instrumentation and techniques for disc space preparation and artificial disc implantation.

### SUMMARY OF THE INVENTION

The invention generally is directed to a distraction instrument for distracting an intervertebral site and to a method of implanting an artificial disc or fusion implant into an intervertebral site.

One embodiment of the invention is a distraction instrument that includes a drive rod, a driver mechanism coupled to the drive rod, and a pair of arms linked to the driver mechanism. The driver mechanism can include a housing adapted to be coupled to the distraction arms and a gear mechanism disposed in the housing for distracting the intervertebral site. The gear mechanism can include a switch having at least two positions, wherein one position provides for a first linear movement of the drive rod and another position disposes the drive rod in a free-floating position. Optionally, the switch can have a position for a second linear movement of the drive rod such as where the first linear movement and the second linear movement are parallel to each other. The gear mechanism can be a ratcheting gear mechanism that includes a first wheel gear and a second wheel gear, wherein the first gear and the second gear are integrated. The drive rod engages the first wheel gear, and a first straight gear engages the second wheel gear, wherein the straight gear is engaged by the switch. In another embodiment, the ratcheting gear mechanism further includes a third wheel gear integrated with the first wheel gear, and a second straight gear engaging the third wheel gear, wherein the switch engages the first straight gear or the second straight gear. In one embodiment, the actuating mechanism is a lever. The drive rod can further include a plurality of teeth extending along a length of the drive rod and optionally a head at an end of the drive rod. In one embodiment, the head of the drive rod has a height in a range of between about 5.0 mm and about 30 mm. The head can include a pair of diametrically opposed wheels. In another embodiment, an implant holder is coupled to the rod at one end.

In a specific embodiment, the distraction instrument of the invention includes a pair of arms, a housing linking the pair of arms, a driver mechanism within the housing, a drive rod engaging the driver mechanism, and an implant holder coupled to the drive rod. Actuation of the gear mechanism causes the implant holder to move to an intervertebral site until the implant holder abuts vertebrae at the intervertebral site, at which point continued actuation of the gear mechanism causes the arms to move away from the intervertebral site, thereby disengaging the distraction instrument from the vertebrae.

In another embodiment, the distraction instrument includes a pair of arms, a housing linking the pair of arms, a gear mechanism within the housing, a drive rod engaging the gear mechanism, and a head at one end of the drive rod. Actuation of the gear mechanism causes the head to move the distraction instrument from a reduced position to a distraction position, thereby causing distraction of vertebrae at an intervertebral site into which a portion of the arms distal to the housing have been inserted.

A method of implanting an artificial disc or a fusion implant in an intervertebral site, includes the steps of preparing an intervertebral site, actuating a gear mechanism of a distraction instrument to distract the intervertebral site, and inserting at least a core of the artificial disc or a spacer of the fusion implant into the intervertebral site. In one embodiment, the gear mechanism is a ratcheting gear mechanism. The gear mechanism can be coupled to a drive rod and can include a switch having at least two positions, wherein one position provides for a first linear movement of the drive rod and another position that disposes the drive rod in free-floating position. The gear mechanism in this embodiment also includes a first wheel gear and a second wheel gear, wherein the first gear and the second gear are integrated, and wherein the first gear engages the drive rod. Also in this embodiment, a first straight gear engages the second wheel gear, wherein the straight gear is engaged or disengaged by the switch.

In one embodiment, the method further includes the step of inserting end plates of an artificial disc or of a fusion implant before distracting the intervertebral site. In this embodiment, the drive rod can include a head, whereby actuating the gear mechanism causes the head to separate the distal ends of arms inserted into the intervertebral site, thereby distracting the intervertebral site.

In an alternate embodiment of the method, the drive rod is coupled to an implant holder, wherein the implant holder is guided along a pair of arms of the distraction instrument. In this embodiment, actuation of the gear mechanism directs the drive rod and implant holder in a first direction until the artificial disc or fusion implant is inserted into the intervertebral site.

In one embodiment, the method further includes the step of removing the distraction instrument from the intervertebral site. One method of removing the distraction instrument includes actuating the gear mechanism. In one embodiment, actuating the gear mechanism to remove the distraction instrument causes movement of the drive rod in the same direction as during distraction of the intervertebral site. In another embodiment, the distraction instrument is removed by moving the switch to a removal position and repeatedly depressing a lever coupled to the gear mechanism until the distraction instrument moves from a distraction position to a reduced position.

The present invention provides many advantages, such as desired distraction without the use of distraction linkage or impaction. Further, the present invention utilizes a gear mechanism to control the amount of distraction to a surgical site. Contrary to some distraction instruments described previously, the gear mechanism of the present invention minimizes impaction of the spine. The use of the gear mechanism of the present invention decreases the amount of required impacts during the procedure, thereby significantly reducing the potential for neural damage.

### BRIEF DESCRIPTION OF THE DRAWINGS

FIG. 1 is a perspective view of the lower spine, highlighting a surgically prepared disc space;

FIG. 2A is a perspective view of an embodiment of the distraction instrument of the invention in a retracted position, wherein the distraction instrument includes distraction arms, drive rod and drive mechanism;

FIG. 2B is a perspective view of the embodiment of FIG. 2A in a distracted position;

FIG. 3A is a perspective view of a driver mechanism of the embodiment of the invention shown at FIG. 2A;

FIG. 3B is a plan view of the embodiment of FIG. 3A;

FIG. 3C is a perspective internal view of one embodiment of the driver mechanism of FIG. 3A;

FIG. 3D is a plan internal view of the embodiment of FIG. 3A;

FIG. 4A is a perspective internal view of another embodiment of the driver mechanism, in a first driving mode;

FIG. 4B is a plan internal view of an embodiment of FIG. 4A;

FIG. 4C is a plan internal view of the embodiment of FIG. 4A in a removal mode;

FIG. 4D is a plan interval view of the embodiment of FIG. 4A in a free-floating mode;

FIG. 5A is a perspective view of another embodiment of a distraction instrument of the present invention in a non-distracted, or reduced, position;

FIG. 5B is a plan view of the distraction instrument of FIG. 5A;

FIG. 6A is a perspective view of the distraction instrument of FIG. 5A with an artificial disc core sandwiched between two implant endplates of the distraction instrument in a distracted position;

FIG. 6B is a plan view of the embodiment of FIG. 6A;

FIG. 7A is a plan view of the distraction arms of the embodiment of FIGS. 6A and 6B, independent of other distraction tools;

FIG. 7B is a perspective view of the distraction arms of FIG. 7A;

FIG. 8A is a perspective view of a v-shaped hinge on the distraction arms of the embodiment of the invention shown in FIGS. 6A and 6B;

FIG. 8B is a close-up view of the v-shaped hinge of FIG. 8A;

FIG. 9A is a perspective view of an embodiment of a drive rod of the distraction instrument of the invention;

FIG. 9B is a close-up plan view of the head portion of the drive rod of FIG. 9A;

FIG. 9C is a close-up perspective view of the head portion of the drive rod of FIG. 9A;

FIG. 9D is a plan view of the drive rod of FIG. 9A;

FIG. 10A is a perspective view of another embodiment of a drive rod of the distraction instrument of the invention;

FIG. 10B is a plan view of the embodiment of FIG. 10A;

FIG. 11A is a perspective view of an embodiment of a distraction mechanism of the present invention at an insertion position;

FIG. 11B is a perspective internal view of the embodiment of FIG. 11A;

FIG. 11C is a perspective view of FIG. 11A in a retracted position; and

FIG. 11D is a plan view of the embodiment of FIG. 11A of the retracted position.

### DETAILED DESCRIPTION OF THE INVENTION

The features and advantages of the invention will be apparent from the following more descriptive explanation of the invention's preferred embodiments, as illustrated in the accompanying drawings. The same number in different figures represents the same item. The drawings are not necessarily to scale, with emphasis instead being placed upon illustrating the principles of the invention.

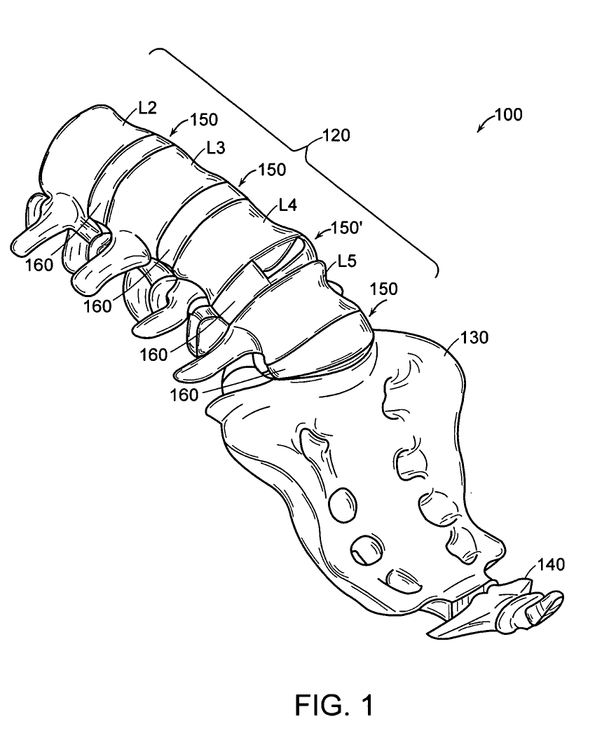

In general, surgical implantation utilizes an anterior approach. During the surgery, a small incision is made in the abdomen below the umbilicus. Internal organs are carefully moved to the side so that the surgeon can visualize the spine. The surgeon then removes a portion of a disc as shown in FIG. 1, a perspective view of the lower region of spine 100. This region comprises lumbar spine 120, sacral spine 130, and coccyx 140. Lumbar spine 120 is comprised of five (5) vertebrae L5, L4, L3, L2, and L1 (not shown). Intervertebral discs 150 link contiguous vertebra from C2 (not shown) to sacral spine 130, wherein a single quotation (′) denotes a damaged disc, for example 150′.

Intervertebral disc 150 is comprised of a gelatinous central portion called the nucleus pulposus (not shown) and surrounded by an outer ligamentous ring called the annulus fibrosus (“annulus”) 160. The nucleus pulposus is composed of 80-90% water. The solid portion of the nucleus is Type II collagen and non-aggregated proteoglycans. Annulus 160 hydraulically seals the nucleus, and allows intradiscal pressures to rise as the disc is loaded. Annulus 160 has overlapping radial bands which allow torsional stresses to be distributed through the annulus under normal loading without rupture.

Annulus 160 interacts with the nucleus. As the nucleus is pressurized, the annular fibers prevent the nucleus from bulging or herniating. The gelatinous nuclear material directs the forces of axial loading outward, and the annular fibers help distribute that force without injury. Damaged disc 150′ is prepared to receive the artificial disc by removing a window the width of the artificial disc to be implanted from annulus 160 of damaged disc 150′. The nucleus pulposus of disc or intervertebral site 150′ is completely removed and ready for distraction procedures.

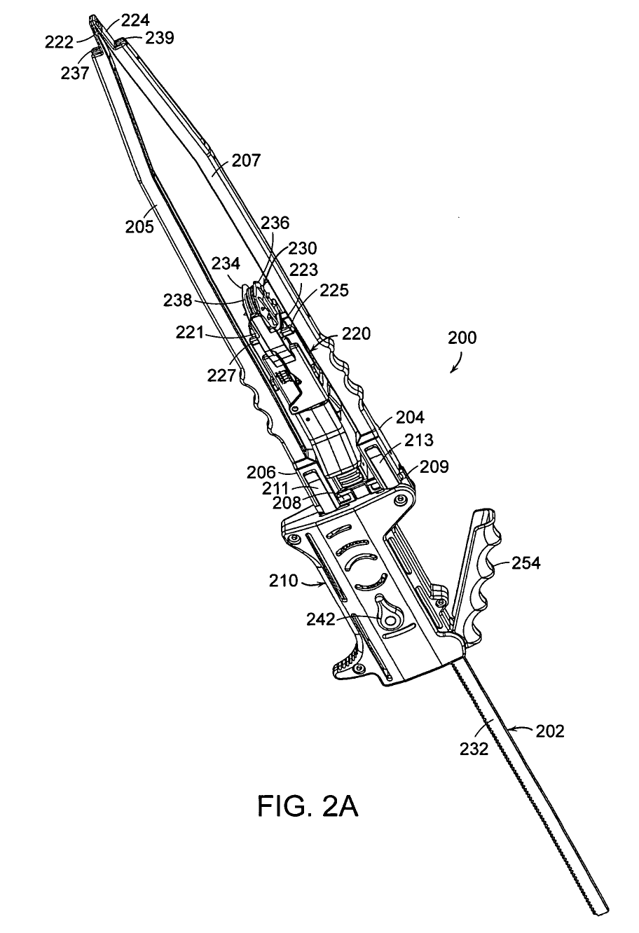

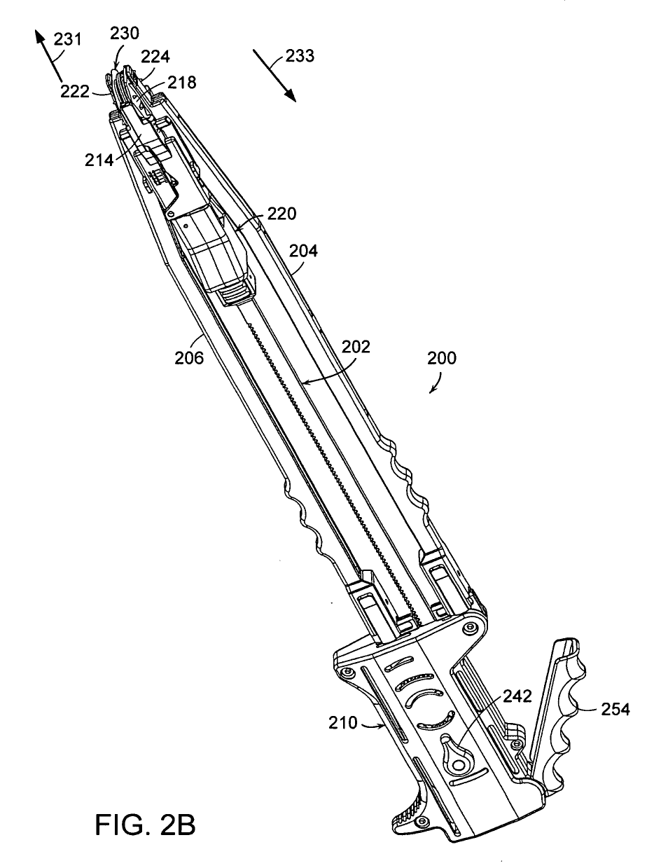

In a first embodiment of the distraction instrument of the present invention, shown in FIGS. 2A and 2B, distraction instrument 200 includes drive rod 202, distraction arms 204, 206 and driver mechanism 210. Driver mechanism 210 operates by a gear mechanism, meaning that the incremental actuating movements of the gears propel drive rod 202 towards or away from intervertebral site 150′ (FIG. 1). In turn, drive rod 202 moves linearly along an axis.

FIG. 2A shows distraction instrument 200 in a reduced position. FIG. 2B shows distraction instrument 200 in a distracted position. In both FIGS. 2A and 2B, drive rod 202 is coupled to implant holder 220, which holds artificial disc 230. Located between arms 204, 206 is drive rod 202. Implant holder 220 is a work piece that is put in motion to transport artificial disc 230 into intervertebral site 150′ (FIG. 1). Implant holder 220 is releasably coupled to both artificial disc 230 and drive rod 202, wherein artificial disc 230 is located at a distal end of implant holder 220 and drive rod 202 is located at a proximal end. Implant holder 220 has guiding feature 221, 223 to align with guiding surfaces 205, 207 of distraction arms 204, 206 as implant holder 220 approaches to thereby insert artificial disc 230 into intervertebral site 150′ (FIG. 1). Artificial disc 230 includes end plates 234, 236 and core 238, which allows movement of end plates 234, 236 relative to each other, and is removably attached to implant holder 220. It is to be understood that, rather than an artificial disc, a fusion implant can be surgically implanted by the apparatus and the method of the invention. A fusion implant can include, for example, endplates and a support member to properly space the endplates.

The proximal end of distraction arms 204, 206 are coupled to driver mechanism 210. Distraction arms 204, 206 are coupled to driver mechanism 210 by a suitable linkage. In one embodiment, shown in FIGS. 2A and 2B, the linkage includes slotted tabs 208, 209 of driver mechanism 210 and clips 211, 213 of distraction arms 204, 206 which lock onto slotted tabs 208, 209. This linkage provides for limited movement of arms 204, 206 along the slots, and limited rotation of arms 204, 206 about the point of linkage with driver mechanism 210. Blades 222, 224 are located at the distal ends 237, 239 of distraction arms 204, 206. Distal ends 237, 239 bear against the boney endplates adjacent to intervertebral site 150′ (FIG. 1) when distraction instrument 200 is in use. Outer surfaces 205, 207 act as guiding surfaces for implant holder 220 during the insertion of artificial disc 230. Distal ends of arms 204, 206 have stops 237, 239 for engaging the anterior surface of intervertebral bodies adjacent to site 150′.

Drive rod 202 includes rack of teeth 232 along at least part of its length, to engage driver mechanism 210. Actuation of driver mechanism 210 by depressing lever 254 drives implant holder 220 towards intervertebral site 150′ (FIG. 1) until artificial disc 230 is located therein. Intervertebral site 150′ is distracted by the force of moving implant holder 220 between distraction arms 204, 206 toward blades 222, 224. When artificial disc 230 is positioned at intervertebral site 150′, implant holder 220 is stopped by abutment of implant holder stop pairs 225, 227 against anterior portions of vertebrae, and no longer moves forward. At this point, the surgeon continues ratcheting movement by repeatedly depressing lever 254. However, since implant holder stops 225, 227 prevent further movement of implant holder 220, distraction arms 204, 206 now move relative to drive rod 202 and implant holder 220 in a direction indicated by arrow 233 and thereby remove blades 222, 224 from the disc space. Withdrawal of blades 222, 224 from the intervertebral site causes adjacent vertebrae to collapse onto artificial disc 230. Pressure of the adjacent vertebrae on artificial disc 230 holds artificial disc 230 in place while the surgeon removes distraction instrument 200 from the surgical site, thereby releasing artificial disc 230 from implant holder 220.

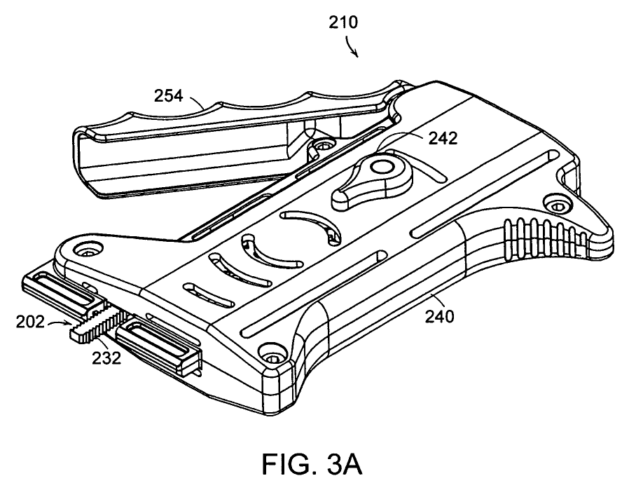

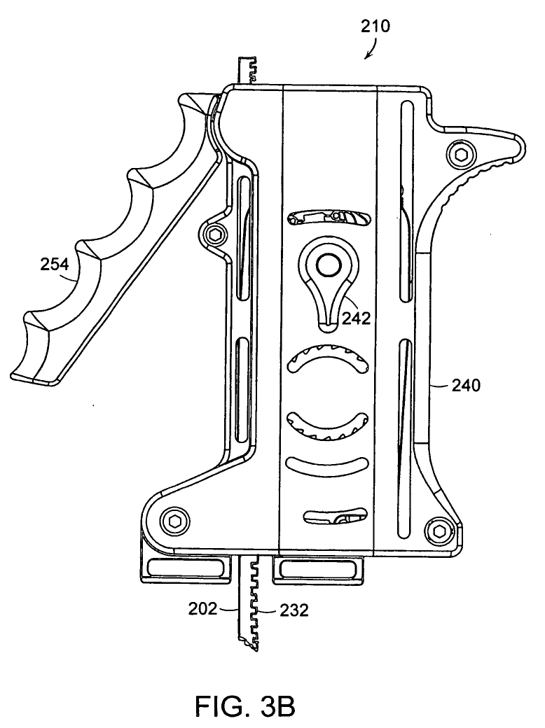

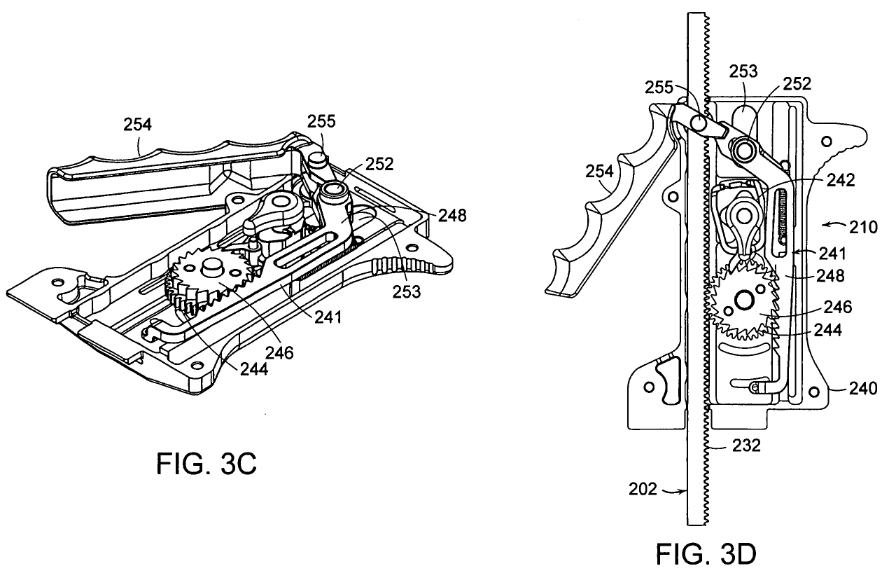

In one embodiment, shown in FIGS. 3A and 3B, housing 240 includes a gear mechanism that is a ratcheting gear mechanism. Referring to FIGS. 3C and 3D, ratcheting gear mechanism 241 includes a gear system that engages rack of teeth 232. Gear mechanism 241 includes two integrated wheel gears 244, 246 and straight gear 248. Straight gear 248 is attached pivotally in housing 240 at post 252. Lever 254 of ratcheting gear mechanism 241 is pivoted at a point 255 near post 252, whereby directing lever 254 toward the remainder of driver mechanism 210 causes post 252 to move along slot 253 in a direction parallel to a major axis of drive rod 202. Actuation of lever 254 thereby translates movement to straight gear 248, which engages wheel gear 246, rotating it counterclockwise. Because wheel gear 246 is also integrated with wheel gear 244, wheel gear 244 also turns counterclockwise and engages rack of teeth 232 of drive rod 202 and moves drive rod 202 towards an intervertebral site. When the surgeon repeatedly depresses lever 254, drive rod 202 is driven incrementally towards the intervertebral site.

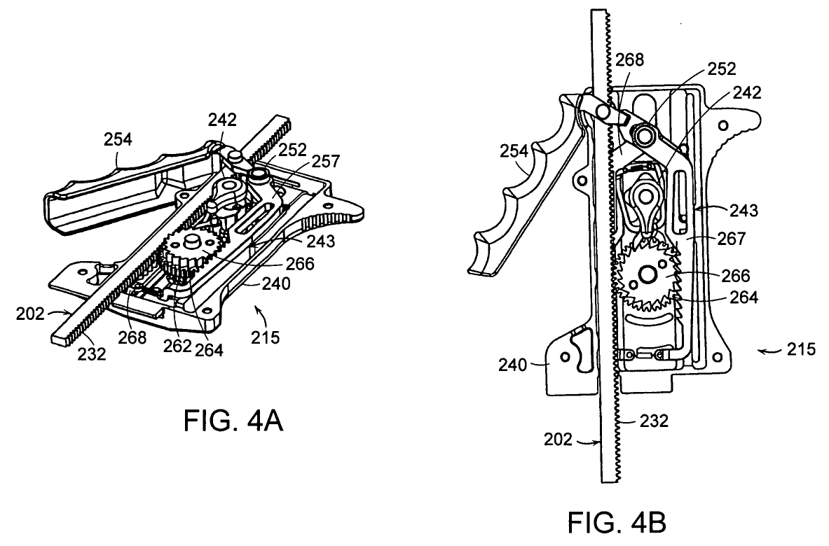

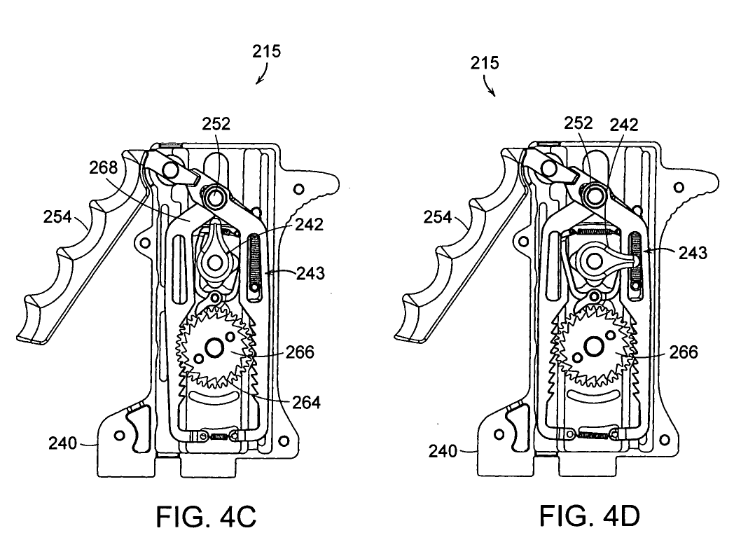

In a second embodiment, ratcheting gear mechanism 243, shown in FIGS. 4A and 4B, includes three wheel gears and two diametrically opposed straight gears 267, 268. The three wheel gears consist of two outer wheel gears 262, 266 and inner wheel gear 264. Each of the outer wheel gears are paired with one of the two straight gears, and only one outer wheel gear-straight gear pair is actuated depending on the mode of driver mechanism 210.

The mode of the ratcheting gear mechanism is controlled by switch 242. In one embodiment, shown in FIGS. 3C and 3D, there are two modes of operation: a driving mode and a free-floating mode. In another embodiment, shown in FIGS. 4A through 4D, the gear mechanism can have three modes of operation: a driving, a removal and a free-floating mode. When switch 242 is set in a driving mode or position, shown in FIGS. 4A and 4B, actuation of lever 254 engages straight gear 267 with outer wheel gear 266, whereby inner wheel gear 264 linearly moves drive rod 202 along an axis. Switch 242 can also be set at a removal position, or mode, shown in FIG. 4C, for removal of drive rod 202, whereby ratcheting movement reverses the movement of drive rod 202 away from the intervertebral site. In this mode, actuation of lever 254 engages straight gear 268 with outer wheel gear 262, thereby retracting drive rod 202. The surgeon can then remove distraction instrument 300 from the surgical site and the patient. The ratcheting gear mechanism of this embodiment also has a free-floating position, or mode, shown in FIG. 4D, wherein straight gears 267 and 268 disengage the wheel gears, allowing drive rod 202 to move freely.

Driver mechanism 210 can be adapted to be integrated with the distraction tools disclosed in U.S. Patent Application No. 2005/0027300, filed Mar. 31, 2004, the entire teachings of which are herein incorporated by reference.

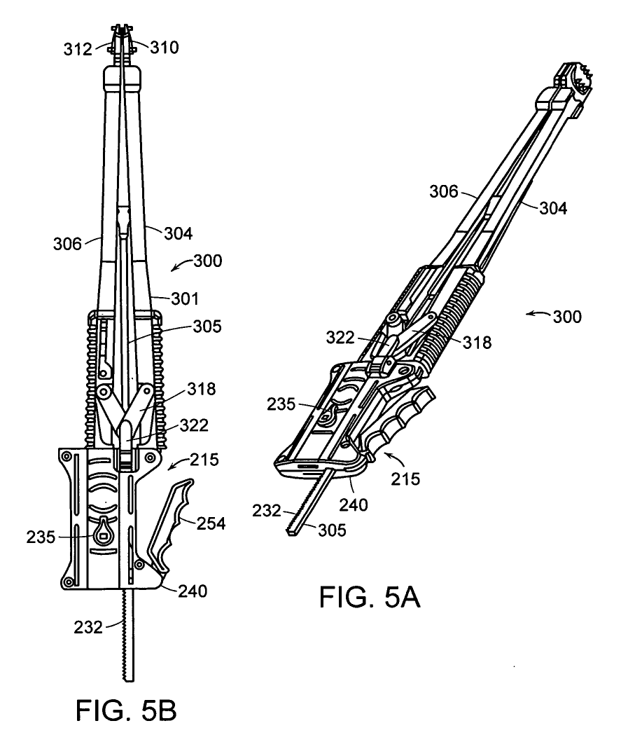

In another embodiment of the distraction instrument of the present invention, shown in FIGS. 5A and 5B, distraction instrument 300 includes drive rod 305, distraction arms 304, 306 and driver mechanism 210. Distraction arms 304, 306 are coupled to driver mechanism 210 and hold end plates 310, 312. Distraction instrument 300 can use the same driver mechanism as distraction instrument 200 of the first embodiment of the present invention.

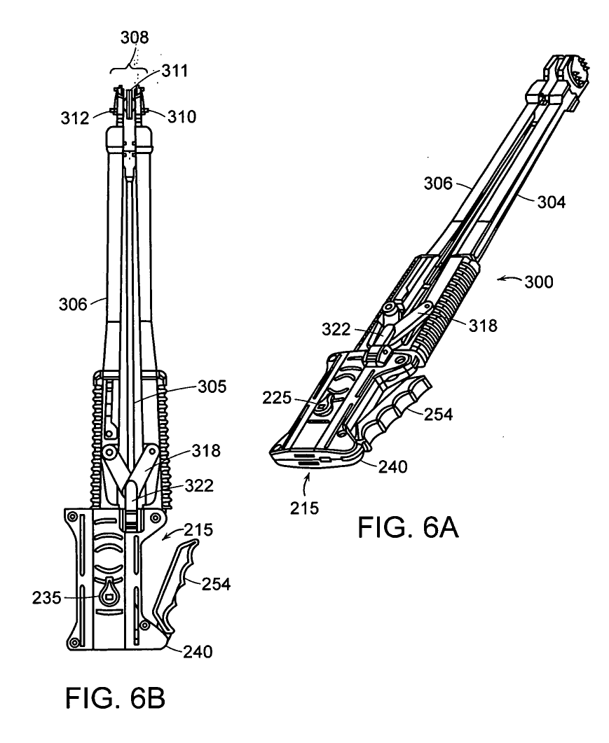

As shown in FIGS. 6A and 6B, core 311 is sandwiched between end plates 310, 312 of implant 308. It is to be understood, however, that, instead of the core of an artificial disc, a supporting spacer can be sandwiched between endplates of a fusion implant. During implantation, the distal ends of distraction arms 304, 306 bear against the anterior faces of vertebral bodies adjacent to site 150′ (FIG. 1). The drive rod touches distraction arms 304, 306, along the internal slots of distraction arms 304, 306. When the surgeon repeatedly depresses lever 254, drive rod 305 is driven incrementally towards intervertebral site 150′ (FIG. 1). As drive rod 305 proceeds from one end of the rails toward intervertebral site 150′ (FIG. 1), distraction arms 304, 306 spread apart. When arms 304, 306 are sufficiently apart, artificial disc core 311 is inserted between end plates 310, 312.

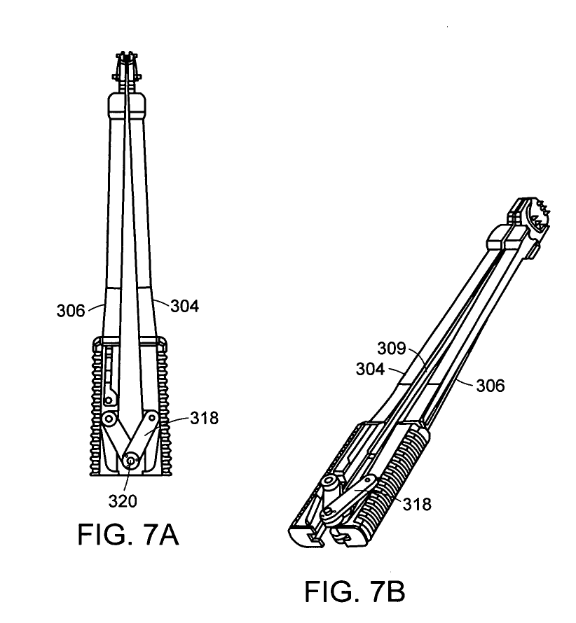

FIGS. 7A and 7B show distraction arms 304, 306 unattached to any driver mechanism 210. Distraction arms 304, 306 are kept apart by v-shaped hinge 318. Hinge 318 has a built-in spring mechanism that returns distraction arms 304, 306 from a collapsed position, wherein distraction arms are essentially parallel, to a desired angular position, shown in FIGS. 7A and 7B.

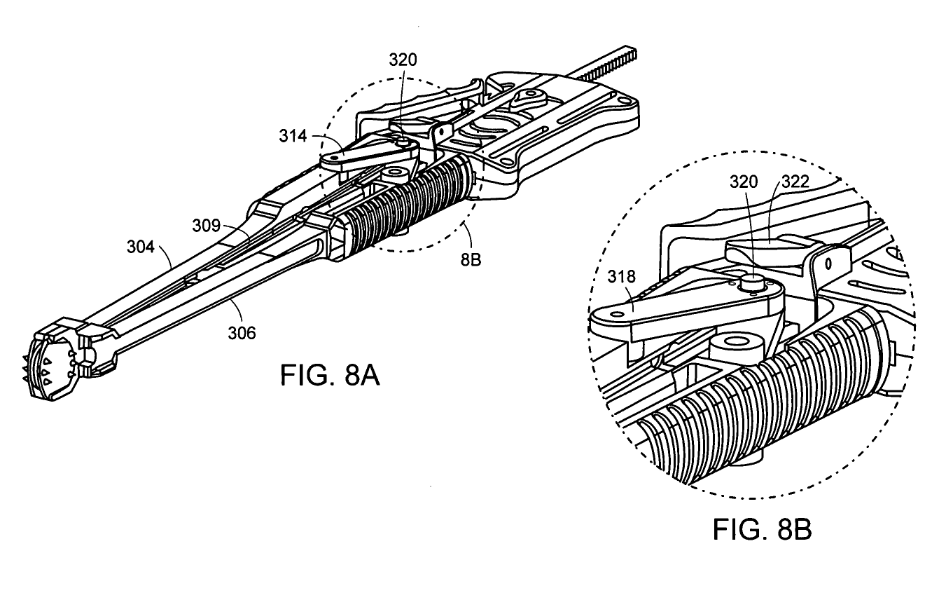

As shown in FIGS. 8A and 8B, hinge 318 is held together by knob 320. Knob 320 also works as a connecting point in conjunction with bridge 322 to couple driver mechanism 210 to distraction arms 304, 306. Bridge 322, pivotally mounted on driver mechanism 210, has a depression (not shown) at its tip. As bridge 322 folds to engage hinge 318, a depression locks onto knob 320, whereby driver mechanism 210 and distraction arms 304, 206 are together immobilized and coupled.

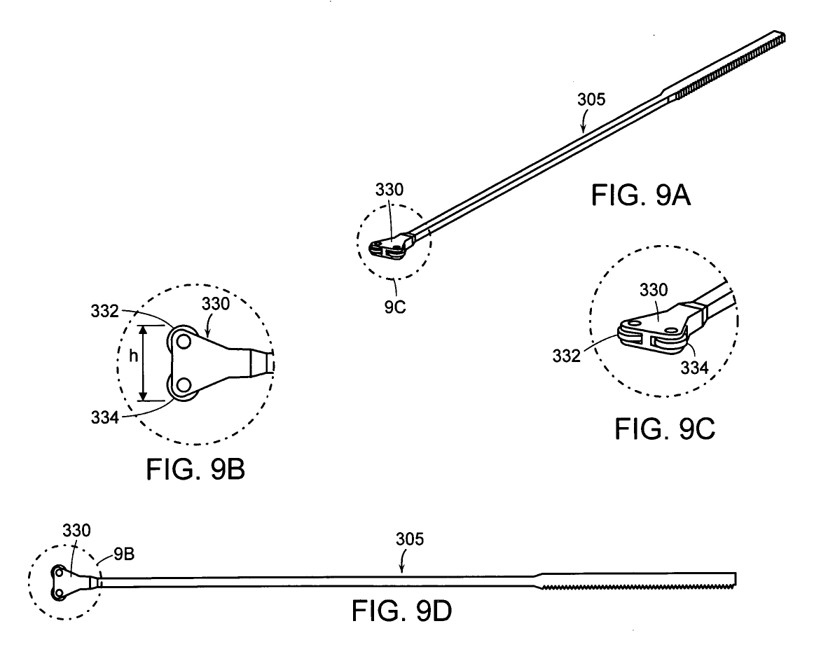

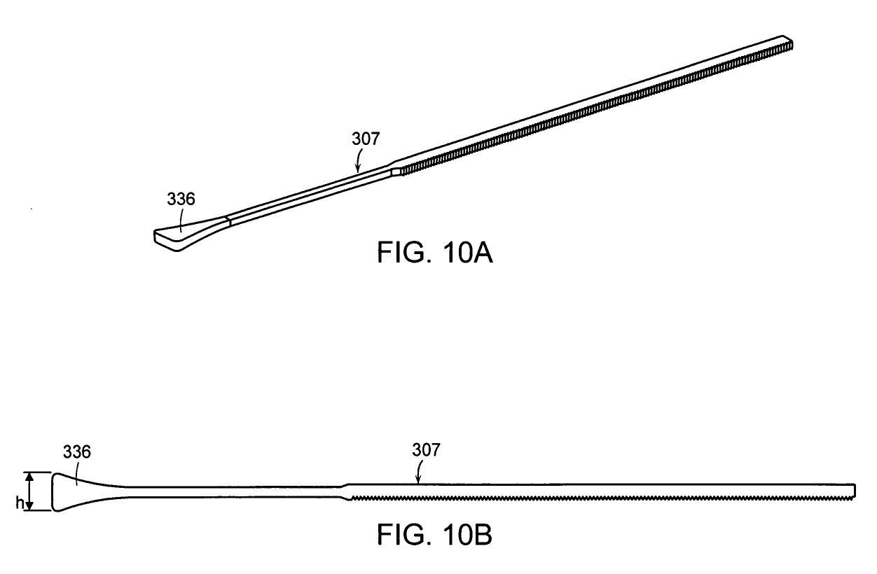

In one embodiment of the drive rod, shown in FIGS. 9A through 9D, head 330 of drive rod 305 has a pair of diametrically opposed wheels 332, 334 that are fitted for and slide along the respective slots (not shown) of arms 304, 306 (FIGS. 8A and 8B). Inner slot surfaces 309 of arms 304, 306 act as guiding surfaces for drive rod 305 during distraction. In another embodiment of the drive rod, shown in FIGS. 10A and 10B, head 336 of drive rod 307 does not have any wheels. Head 336 has a height (“h” in FIG. 9B) from 5.0 mm to 30 mm. Furthermore, as the intervertebral site is distracted by the force of driving drive rod 305 or drive rod 307 between distraction arms 304, 306 (FIG. 8A and 8B), the height “h” of head 330 or 336 determines how much the intervertebral site is distracted. Both drive rod 305 and drive rod 307 have a rack of teeth which engages driver mechanism 210.

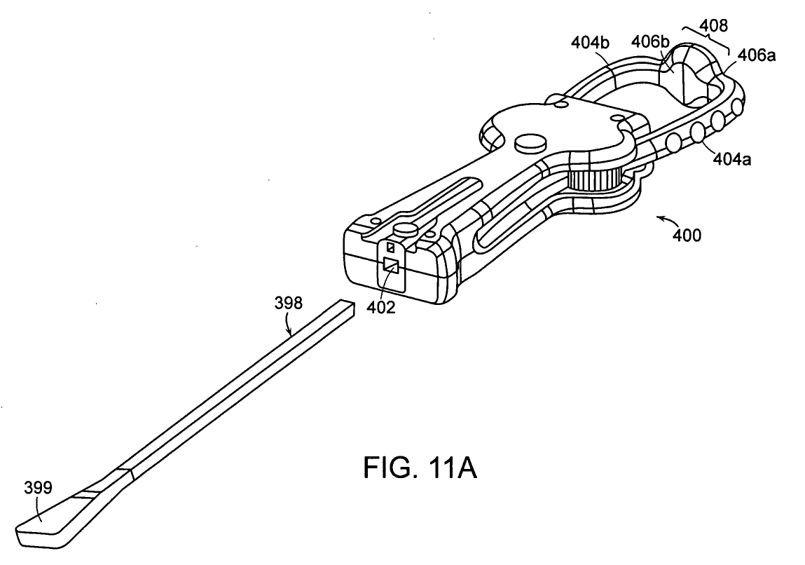

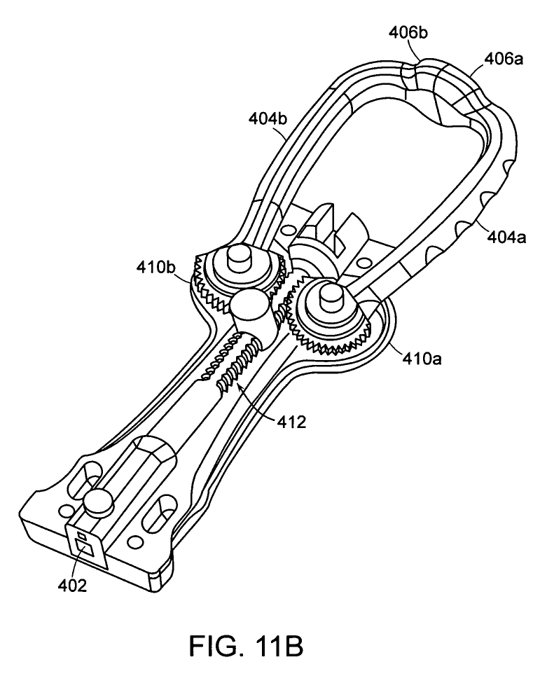

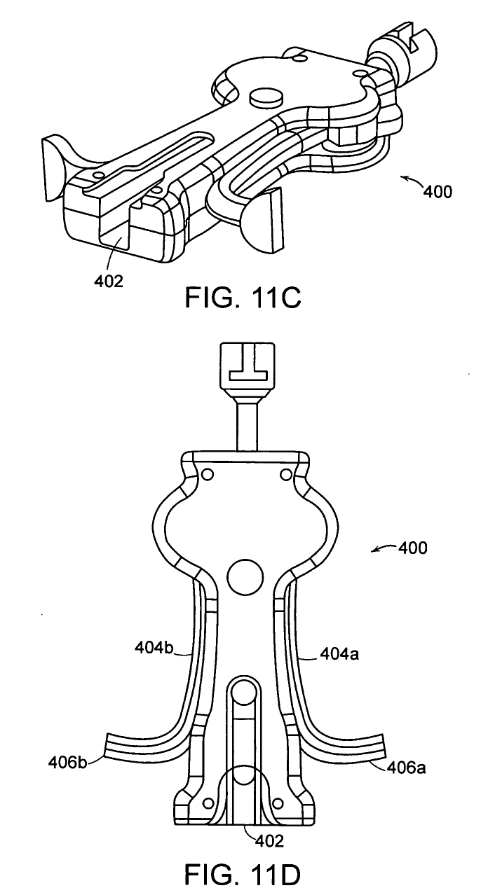

FIGS. 11A through 11D show another embodiment of the gear mechanism and actuating mechanics of a distraction instrument of the present invention. In this embodiment, and referring to FIGS. 7A and 7B, endplates of an artificial disc or a fusion implant, attached to distraction arms 304, 306, coupled by hinge 318, are inserted into an intervertebral site. Drive rod 398 (FIG. 11A), having drive rod head 399, is coupled to driver mechanism 400 at the modular connection 402. Modular connection 402 is a component of gear mechanism 412 shown in FIG. 11B. Gear mechanism 412 includes wheel gears 410a and 410b, arms 404a and 404b, and halves 406a and 406b. Halves 406a and 406b collectively form head 408.

While gear mechanism 412 and the actuating mechanism are in a first position, shown in FIGS. 11A and 11B, the surgeon impacts head 408 to drive rod head 399 down the length of distraction arms 304, 306, thereby distracting the intervertebral site to provide a space between endplates held by distraction arms 304, 306. The surgeon can then insert an artificial disc core or a fusion implant spacer between the endplates. Once the disc core is in place between the endplates, the activating mechanism is moved from the first, or driving, position, shown in FIGS. 11A and 11B, to a second, or removal, position, shown in FIGS. 11C and 11D. Actuation of the driver mechanism from the first to the second position causes wheel gears 410a and 410b to direct gear mechanism 412 away from the surgical site, thereby retracting drive rod 398, which in turn allows distraction arms 304, 306 to collapse and, consequently, the endplates to rest on either side of the artificial disc core. Distraction arms 304, 306 can then be withdrawn from the surgical site by the surgeon, thereby dislodging the endplates from distraction arms 304, 306 and completing implantation of the artificial disc.

While this invention has been particularly shown and described with references to preferred embodiments thereof, it will be understood by those skilled in the art that various changes in form and details may be made therein without departing from the scope of the invention encompassed by the appended claims.

What is claimed is:

1. A distraction instrument for distracting an intervertebral site, comprising;
a) a drive rod;
b) a driver mechanism coupled to the drive rod; and
c) a pair of arms linked to the drive mechanism.

2. The instrument of claim 1, wherein the driver mechanism includes:
a) a housing adapted to be coupled to the distraction arms; and
b) a gear mechanism disposed in the housing for distracting the intervertebral site.

3. The instrument of claim 1, wherein the gear mechanism includes a switch having at least two positions, wherein one position provides for a first linear movement of the drive rod and another position disposes the drive rod in a free floating position.

4. The instrument of claim 3, wherein the switch has a position for a second linear movement of the drive rod.

5. The instrument of claim 3, wherein the first linear movement and the second linear movement are parallel to each other.

6. The instrument of claim 3, wherein the gear mechanism is a ratcheting gear mechanism, comprising:
a) a first wheel gear and a second wheel gear, wherein the first wheel gear and the second wheel gear are integrated;
b) the drive rod engaging the first wheel gear; and
c) a first straight gear engaging the second wheel gear, wherein the straight gear is engaged by the switch.

7. The instrument of claim 5, wherein the ratcheting gear mechanism further includes:
a) a third wheel gear integrated with the first wheel gear; and
b) a second straight gear engaging the third wheel gear, wherein the switch engages the first straight gear or the second straight gear.

8. The instrument of claim 1, wherein the actuating mechanism is a lever.

9. The instrument of claim 1, wherein the drive rod further includes a plurality of teeth extending along a length of the drive rod.

10. The instrument of claim 1, wherein the drive rod further includes a head at an end of the drive rod.

11. The instrument of claim 10, wherein the head has a height in a range of between about 5.0 mm and about 30 mm.

12. The instrument of claim 10, wherein the head includes a pair of diametrically opposed wheels.

13. The instrument of claim 10 further including an implant holder coupled to the rod.

14. A distraction instrument comprising:
a) a pair of arms;
b) a housing linking the pair of arms;
c) a driver mechanism within the housing;
d) a drive rod engaging the driver mechanism; and
e) an implant holder coupled to the drive rod,
whereby actuation of the gear mechanism causes the implant holder to move toward an intervertebral site until the implant holder abuts vertebral bodies of the intervertebral site, at which point continued actuation of the gear mechanism causes the arms to move away from the intervertebral site, thereby disengaging the distraction instrument from the vertebral bodies.

15. A distraction instrument comprising:
a) a pair of arms;
b) a housing linking the pair of arms;
c) a gear mechanism within the housing;
d) a drive rod engaging the gear mechanism; and
e) a head at one end of the drive rod, whereby actuation of the gear mechanism causes the head to move the distraction instrument from a reduced position to a distraction position, thereby causing distraction of vertebrae at an intervertebral site into which a portion of the arms distal to the housing have been inserted.

16. A method of implanting an artificial disc or a fusion implant in an intervertebral site, comprising the steps of:
a) preparing an intervertebral site;
b) actuating a gear mechanism of a distraction instrument to distract the intervertebral site; and
c) inserting at least a core of the artificial disc or a spacer of the fusion implant into the intervertebral site.

17. The method of claim 16, wherein the gear mechanism is a ratcheting gear mechanism.

18. The method of claim 17, wherein the gear mechanism is coupled to a drive rod and comprises:
a) a switch having at least two positions, wherein one position provides for a first linear movement of the drive rod and another position that disposes the drive rod in a free floating position;
b) a first wheel gear and a second wheel gear, wherein the first gear and the second gear are integrated, and wherein the first gear engages the drive rod; and
c) a first straight gear engaging the second wheel gear, wherein the straight gear is engaged or disengaged by the switch.

19. The method of claim 18, further including the step of inserting end plates of the artificial disc or of the fusion implant before distracting the intervertebral site.

20. The method of claim 19, wherein the drive rod includes a head, whereby actuating the gear mechanism causes the head to separate ends of arms inserted into the intervertebral site, thereby distracting the intervertebral site.

21. The method of claim 18, wherein the drive rod is coupled to an implant holder, that is holding an artificial disc or fusion implant, wherein the implant holder is guided along a pair of arms of the distraction instrument.

22. The method of claim 21, further including continually actuating the ratcheting gear mechanism to direct the drive rod and implant holder in a first direction until the artificial disc or fusion implant is inserted in the intervertebral site.

23. The method of claim 16, further including the step of removing the distraction instrument from the intervertebral site.

24. The method of claim 23, wherein removing the distraction instrument includes actuating the gear mechanism.

25. The method of claim 24, wherein actuating the gear mechanism to remove the distraction instrument causes movement of the drive rod in the same direction as during distraction of the intervertebral site.

26. The method of claim 24, wherein removing the distraction instrument includes the steps of:
a) moving the switch to a removal position; and
b) repeatedly depressing a lever coupled to the gear mechanism until the distraction instrument moves from a distraction position to a reduced position.
